(19) United States
(12) Patent Application Publication
(10) Pub. No.: US 2023/0330411 A1
(43) Pub. Date: Oct. 19, 2023

(54) CLOSE-CONTACT TYPE NERVE STIMULATOR

(71) Applicant: NEURIVE Co., Ltd., Gimbae-si, Gyeongsangnam-do (KR)

(72) Inventor: Jae Jun SONG, Seoul (KR)

(21) Appl. No.: 18/023,451

(22) PCT Filed: Aug. 28, 2020
(86) PCT No.: PCT/KR2020/011534

§ 371 Date: Feb. 27, 2023

(51) Int. Cl. A61N 1/04

(52) U.S. Cl. CPC A61N 1/0456; A61N 1/0476

(57) ABSTRACT

Proposed is a close-contact type nerve stimulator. The nerve stimulator includes a fixed part which is disposed across the front and rear of an ear, a body part which is rotatably connected to the fixed part, an electrode part which is provided in the body part and is configured to be brought into contact with the auricle or external auditory meatus of the ear, and a control part which controls the angle of rotation of the body part with respect to the fixed part, wherein the electrode part may be brought into close contact with the surface of the auricle or external auditory meatus by the rotation of the body part so as to stimulate the vagus nerve distributed in the auricle or external auditory meatus.

### TECHNICAL FIELD

The present disclosure relates to a close-contact type nerve stimulator and, more particularly, to a close-contact type nerve stimulator capable of making close contact with the surface of the auricle or external auditory meatus by having an electrode whose angle may be adjusted and that is deformable according to the shape and size of the auricle or external auditory meatus.

### BACKGROUND ART

The vagus nerve is one of the cranial nerves and runs from the brain through the face and thorax (chest) to the abdomen. As such, the vagus nerve, which is distributed to many important organs, can performs a variety of functions such as controlling the pharyngeal constrictor muscles and the intrinsic laryngeal muscles, and regulating heart rate, intestinal peristalsis, and sweat secretion.

Therefore, by stimulating the vagus nerve, it is possible to treat various conditions such as tinnitus, dementia, cognitive impairment, degenerative brain disease, and chronic pain in addition to depression and epilepsy.

In order to treat these conditions, a method of attaching and transplanting an electrical stimulator to the vagus nerve through surgery is used.

The above-described background art is possessed or acquired by the inventor in the process of deriving the present disclosure, and cannot necessarily be said to be known art disclosed to the general public prior to filing the present disclosure.

### DISCLOSURE

### Technical Problem

The present disclosure has been made keeping in mind the problems occurring in the related art. An objective of the present disclosure is to provide a close-contact type nerve stimulator that effectively stimulates the vagus nerve distributed in the auricle or external auditory meatus with an electrode in close contact with the surface of the auricle or external auditory meatus by having the electrode whose angle may be adjusted and that is deformable according to the shape and size of the auricle or external auditory meatus.

Objectives of the present disclosure are not limited to the objective mentioned above, and other objectives not mentioned will be clearly understood by those skilled in the art from the description below.

### Technical Solution

In order to achieve the above mentioned objective, according to an embodiment of the present disclosure, there is provided a close-contact type nerve stimulator. The nerve stimulator includes: a fixed part disposed across front and rear of an ear; a body part rotatably connected with respect to the fixed part; an electrode part installed on the body part and configured to contact auricle or external auditory meatus; and a control part configured to adjust an angle of rotation of the body part with respect to the fixed part, wherein the electrode part may come into close contact with a surface of the auricle or the external auditory meatus by a rotation of the body part so as to stimulate the vagus nerve distributed in the auricle or the external auditory meatus.

According to the embodiment of the present disclosure, the control part may include: a first adjustment member provided as a shaft that hinge couples the fixed part and the body part; a second adjustment member disposed on either one of the fixed part or the body part, and configured to slide toward a remaining one; and a third adjustment member disposed on a remaining one of the fixed part or the body part, and famed to come into contact with the second adjustment member, wherein the third adjustment member may rotate, by a sliding of the second adjustment member, one of the fixed part and the body part relative to the remaining one with the first adjustment member as a center.

According to the embodiment of the present disclosure, the third adjustment member may include: an internal space in which a portion of the second adjustment member is accommodated; and an inclined surface to which a portion of the second adjustment member contacts, wherein the portion of the second adjustment member may be slidable along the inclined surface, and the inclined surface may be rotated around the first adjustment member.

According to the embodiment of the present disclosure, when the second adjustment member moves from one of the fixed part and the body part toward the remaining one, an angle between the fixed part and the body part may decrease, whereas when the second adjustment member moves from the remaining one of the fixed part and the body part toward the one, the angle between the fixed part and the body part may increase.

According to the embodiment of the present disclosure, the body part may be capable of elastically restoring to an original position thereof.

According to the embodiment of the present disclosure, the electrode part may include a first electrode in contact with the external auditory meatus, wherein the first electrode may be flexibly deformed according to a size or shape of the external auditory meatus so as to be in close contact with the surface of the external auditory meatus.

According to the embodiment of the present disclosure, the first electrode may include: a contact member in contact with the external auditory meatus; and deformable members configured to form spaces at regular intervals outside the contact member, wherein the deformable members may be provided to be stretchable when the contact member contacts the external auditory meatus by the rotation of the body part, and the contact member may be flexibly deformed according to the shape of the external auditory meatus by an expansion and contraction of the deformable members, and may come into close contact with the surface of the external auditory meatus.

According to the embodiment of the present disclosure, the electrode part may further include a second electrode installed at a position on the body part contactable with the tragus.

According to the embodiment of the present disclosure, the electrode part may further include a third electrode installed at a position on the body part contactable with the cavum concha.

According to the embodiment of the present disclosure, the electrode part may further include a fourth electrode installed at a position on the body part contactable with the cymba concha.

In order to achieve the above mentioned objective, according to an embodiment of the present disclosure, there is provided a close-contact type nerve stimulation electrode. The electrode includes: a body part located in auricle; a first electrode installed at a position on the body part contactable with external auditory meatus; a second electrode installed at a position on the body part contactable with the tragus; a third electrode installed at a position on the body part contactable with the cavum concha; and a fourth electrode installed at a position on the body part contactable with the cymba concha, wherein at least one of the first electrode, the second electrode, the third electrode, or the fourth electrode may be flexibly deformed according to a size or shape of the auricle or the external auditory meatus, and may come into close contact with a surface of the auricle or the external auditory meatus to effectively stimulate the vagus nerve.

According to the embodiment of the present disclosure, the at least one of the first electrode, the second electrode, the third electrode, or the fourth electrode may include: a contact member in contact with the auricle or the external auditory meatus; and detonable members configured to font spaces at regular intervals outside the contact member, wherein the deformable members may be provided to be stretchable when the contact member contacts the auricle or the external auditory meatus by a rotation of the body part, and the contact member may be flexibly defamed according to the shape of the auricle or the external auditory meatus by an expansion and contraction of the deformable members, and may come into close contact with the surface of the auricle or the external auditory meatus.

### Advantageous Effects

A close-contact type nerve stimulator according to an embodiment of the present disclosure can effectively stimulate the vagus nerve distributed in the auricle or external auditory meatus with an electrode in close contact with the surface of the auricle or external auditory meatus by having the electrode whose angle can be adjusted and that can be deformed according to the shape and size of the auricle or external auditory meatus.

The effects of the close-contact type nerve stimulator according to the embodiment of the present disclosure are not limited to those mentioned above, and other effects not mentioned will be clearly understood by those skilled in the art from the description below.

### DESCRIPTION OF DRAWINGS

FIG. 1 is an overall perspective view of a close-contact type nerve stimulator according to an embodiment;

FIG. 2 shows the left unit of the close-contact type nerve stimulator according to the embodiment;

FIG. 3 is a cross-sectional view of a control part of the close-contact type nerve stimulator according to the embodiment;

FIG. 4 shows a state in which a body part is rotated by sliding of a first adjustment member of the control part of the close-contact type nerve stimulator according to the embodiment;

FIG. 5 shows an electrode part of the close-contact type nerve stimulator according to the embodiment; and

FIG. 6 is a first electrode of the close-contact type nerve stimulator according to the embodiment.

The following drawings attached to this specification illustrate a preferred embodiment of the present disclosure, and serve to further understand the technical idea of the present disclosure together with the detailed description of the present disclosure. Therefore, the present disclosure should not be construed as being limited only to the matters described in such drawings.

### BEST MODE

Hereinafter, embodiments of the present disclosure will be described in detail through exemplary drawings. It should be noted that, in assigning reference numerals to the components of each drawing, the same reference numerals are given to the same components as much as possible, even if they are shown on different drawings. In addition, in describing an embodiment, when it is determined that a detailed description of a related known configuration or function hinders understanding of the embodiment, the detailed description will be omitted.

In describing the components of the embodiments, terms such as first, second, A, B, (a), and (b) may be used. These terms are only used to distinguish a component from other components, and the nature, order, or order of the component is not limited by the term. It should be understood that, when a component is referred to as being “connected”, “joined”, or “coupled” to another component, that component may be directly connected or coupled to the other component, but another component may be “connected”, “joined”, or “coupled” in between.

Components included in one embodiment and components having common functions will be described using the same names in other embodiments. Unless otherwise stated, descriptions provided in one embodiment may be applied to other embodiments, and detailed descriptions will be omitted to the extent of overlapping.

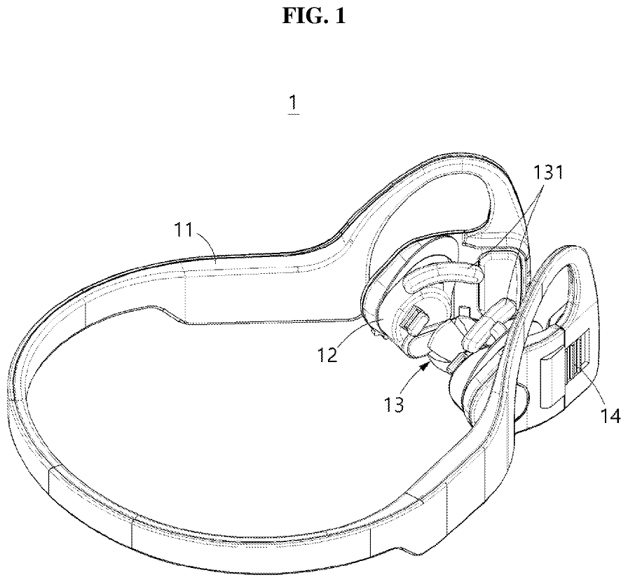

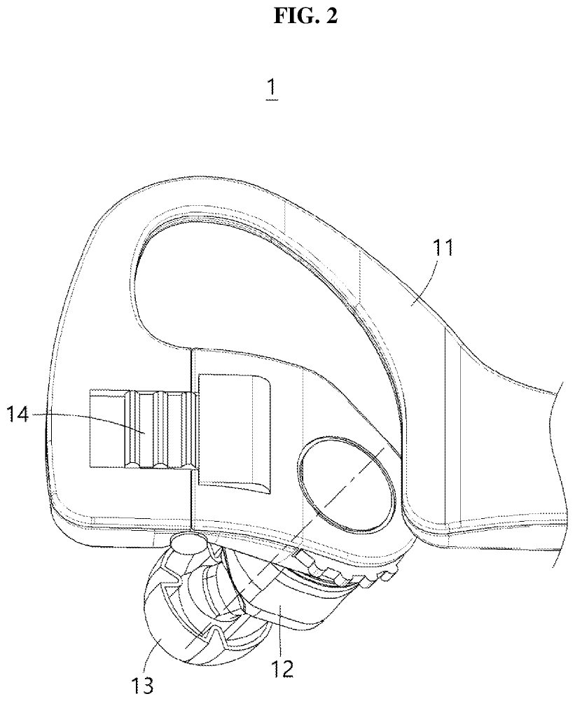

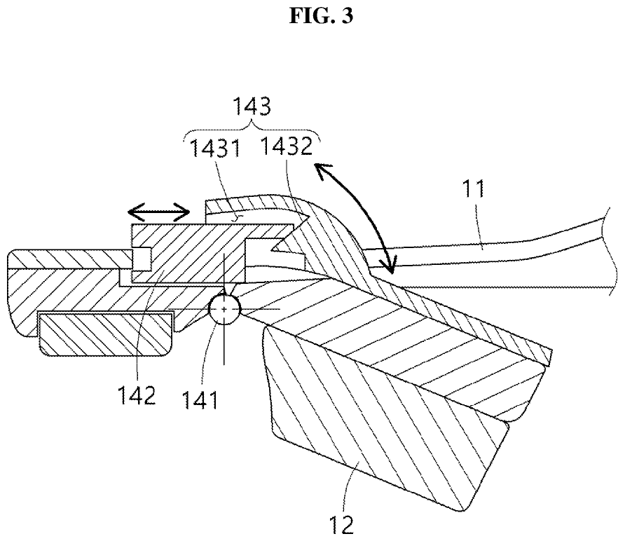

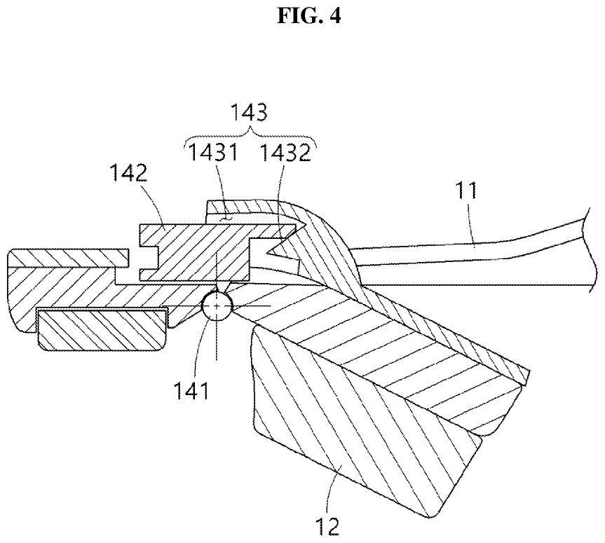

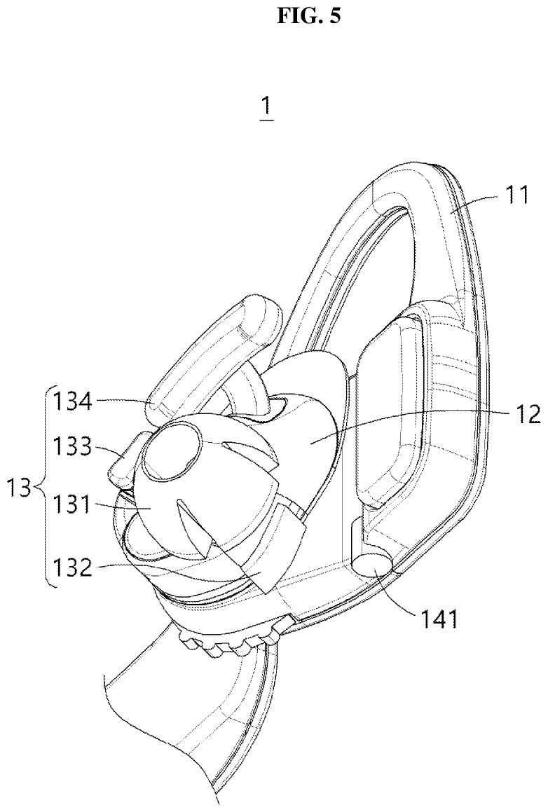

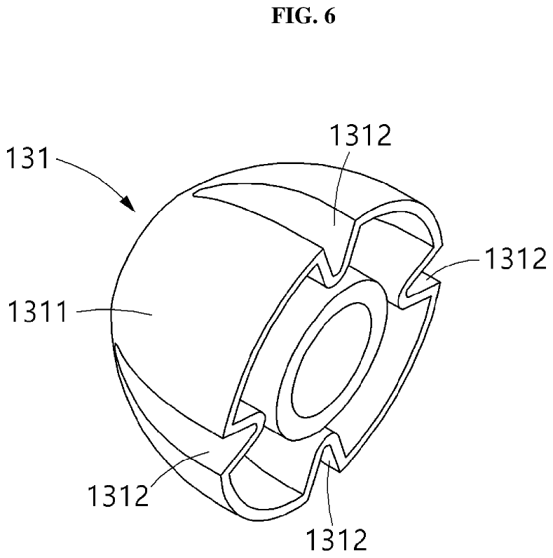

FIG. 1 is an overall perspective view of a close-contact type nerve stimulator 1 according to an embodiment, FIG. 2 shows the left unit of the close-contact type nerve stimulator 1 according to the embodiment, FIG. 3 is a cross-sectional view of a control part 14 of the close-contact type nerve stimulator 1 according to the embodiment, FIG. 4 shows a state in which a body part 12 is rotated by sliding of a first adjustment member 141 of the control part 14 of the close-contact type nerve stimulator 1 according to the embodiment, FIG. 5 shows an electrode part 13 of the close-contact type nerve stimulator 1 according to the embodiment, and FIG. 6 is a first electrode 131 of the close-contact type nerve stimulator 1 according to the embodiment.

Referring to FIGS. 1 and 2, the close-contact type nerve stimulator 1 according to the embodiment may include a fixed part 11, the body part 12, the electrode part 13, and the control part 14.

To be specific, the fixed part 11 may be disposed across the front and rear of an ear. For example, the fixed part 11 may extend in a band shape surrounding the back of the head. In addition, both ends of the fixed part 11 are foiled in a hook shape so as to be stably hung over the ears.

The body part 12 may be connected to each end of the fixed part 11, respectively, and may be rotatably connected with respect to the fixed part 11. In addition, each of the body parts 12 may be respectively located in the left and right auricles.

The electrode part 13 may be installed on each of the left and right body parts 12, and may be configured to contact the left and right auricles or external auditory meatus. At this time, the electrode part 13 may stimulate the vagus nerve distributed in the auricle or the external auditory meatus in a state of close contact with the surface of the auricle or the external auditory meatus by rotation of the body part 12. The electrode part 13 will be described in more detail with reference to FIGS. 5 and 6 later.

The control part 14 may adjust the rotation angle of the body part 12 with respect to the fixed part 11.

Referring to FIG. 3, the control part 14 may include a first adjustment member 141, a second adjustment member 142, and a third adjustment member 143.

The first adjustment member 141 may be provided as a shaft that hinge couples the fixed part 11 and the body part 12.

The second adjustment member 142 may be disposed on either one of the fixed part 11 or the body part 12 and slide toward the other one.

The third adjustment member 143 may be disposed on the other one of the fixed part 11 or the body part 12, and a portion thereof may be famed to come into contact with the second adjustment member 142.

By the sliding of the second adjustment member 142, the third adjustment member 143 may rotate one of the fixed part 11 and the body part 12 relative to the other one with the first adjustment member 141 as the center.

For example, when the second adjustment member 142 is disposed on the fixed part 11 and the third adjustment member 143 is disposed on the body part 12, the second adjustment member 142 may slide on the surface of the fixed part 11 from the fixed part 11 toward the body part 12. The portion of the third adjustment member 143 that the second adjustment member 142 was in contact with is pushed by the second adjustment member 142, so that the third adjustment member 143 may be rotated around the first adjustment member 141. Accordingly, the body part 12 to which the third adjustment member 143 is fixed may also be rotated together.

Hereinafter, a case in which the second adjustment member 142 and the third adjustment member 143 are disposed as in the description above will be described as an example.

Referring to FIGS. 3 and 4, the third adjustment member 143 may include an internal space 1431 and an inclined surface 1432.

The internal space 1431 may accommodate a portion of the second adjustment member 142. At this time, the second adjustment member 142 may be formed in a structure in which one side protrudes. The protruding portion may protrude from the fixed part 11 toward the body part 12. The internal space 1431 is a space where the protruding portion of the second adjustment member 142 may move therein, and the internal space 1431 may be foiled to have a size that does not interfere with the sliding movement of the second adjustment member 142.

The inclined surface 1432 may contact a portion of the second adjustment member 142, that is, the protruding portion of the second adjustment member 142. The inclined surface 1432 may be formed to increase in height along the sliding direction of the second adjustment member 142. For example, the inclined surface 1432 may have a shape in which a height increases along a direction from the fixed part 11 toward the body part 12.

The protruding portion of the second adjustment member 142 may slide along the inclined surface 1432. At this time, as shown in FIG. 3, the second adjustment member 142 may make a translational motion in the direction of the arrow along the surface of the fixed part 11, and the inclined surface 1432 may be rotated in the direction of the arrow with the first adjustment member 141 as the center so that the protruding portion may move.

To be specific, when the second adjustment member 142 moves from the fixed part 11 toward the body part 12, the body part 12 may be rotated in a direction in which an angle between the fixed part 11 and the body part 12 decreases as shown in FIG. 4.

Conversely, when the second adjustment member 142 of FIG. 4 moves back from the body part 12 toward the fixed part 11, the body part 12 may be rotated in a direction in which an angle between the fixed part 11 and the body part 12 increases. That is, the body part 12 may be rotated to return to the position shown in FIG. 3.

Although in the above example, the case where the second adjustment member 142 is disposed on the fixed part 11 and the third adjustment member 143 is disposed on the body part 12 has been described, it is also possible that the control part 14 of the close-contact type nerve stimulator 1 according to the embodiment has a structure in which the second adjustment member 142 is disposed on the body part 12 and the third adjustment member 143 is disposed on the fixed part 11.

In addition, the body part 12 may be provided to be elastically restored to its original position. Accordingly, the body part 12 may be automatically rotated toward the original position when the second adjustment member 142 slides from the body part 12 toward the fixed part 11.

Hereinafter, the electrode part 13 of the close-contact type nerve stimulator 1 according to the embodiment will be described in detail with reference to FIGS. 5 and 6.

Referring to FIG. 5, the electrode part 13 may include a first electrode 131, a second electrode 132, a third electrode 133, and a fourth electrode 134.

The first electrode 131 may be inserted into and brought into contact with the external auditory meatus. The first electrode 131 may be flexibly deformed according to the size or shape of the external auditory meatus. For example, the first electrode 131 may be made of an elastic material. Thus, the first electrode 131 may be inserted into the external auditory meatus and apply electrical stimulation to the surface thereof in a state of close contact.

To be specific, the first electrode 131 may include a contact member 1311 and a deformable member 1312 as shown in FIG. 6.

The contact member 1311 may contact the external auditory meatus. The contact member 1311 may be formed, for example, in a hemispherical shape, and a through hole into which the body part 12 may be inserted may be formed in the center thereof.

A plurality of deformable members 1312 may be provided outside the contact member 1311. Each deformable member 1312 may form a space at regular intervals outside the contact member 1311.

The deformable member 1312 is flexible so as to be deformed according to the size and shape of a user's external auditory meatus when inserted into the external auditory meatus. Accordingly, the contact member 1311 may be flexibly deformed according to the size and shape of the external auditory meatus, and may apply electrical stimulation while being in close contact with the surface of the external auditory meatus.

The second electrode 132 is installed at a position on the body part 12 where the second electrode 132 may come into contact with the tragus and may apply electrical stimulation to the tragus in contact.

The third electrode 133 is installed at a position where the third electrode 133 may come into contact with the cavum concha on the body part 12 and may apply electrical stimulation to the cavum concha in contact.

The fourth electrode 134 is installed at a position where the fourth electrode 134 may come into contact with the cymba concha on the body part 12 and may apply electrical stimulation to the cavum concha in contact.

Although the shape is different from that of the first electrode 131, the second electrode 132, the third electrode 133, and the fourth electrode 134 may also be flexibly deformed according to the size or shape of the tragus, the cavum concha, and the cymba concha. Accordingly, the second electrode 132, the third electrode 133, and the fourth electrode 134 are also brought into close contact with the surfaces of the tragus, the cavum concha, and the cymba concha to effectively stimulate the vagus nerve.

The close-contact type nerve stimulator 1 according to the above-described embodiment is provided with the electrode part 13 that may be angle-adjustable and deformable according to the shape and size of the auricle or external auditory meatus, which is different for each user, in order to adhere the electrode part 13 to the auricle or external auditory meatus, so that the vagus nerve may be effectively stimulated in a state in which the electrode part 13 is in close contact with the surface of the auricle or external auditory meatus.

As described above, the embodiments of the present disclosure have been described by specific details such as specific components and limited embodiments and drawings, but this is only provided to help a more general understanding of the present disclosure, and the present disclosure is not limited to the above embodiments, and various modifications and variations may be made from these descriptions by those having ordinary knowledge in the field to which the present disclosure pertains. For example, appropriate results may be achieved even if the described techniques are performed in an order different from the described method, and/or components such as the described structure, device, etc. are coupled or combined in a form different from the described method, or substituted or substituted by other elements or equivalents. Therefore, the spirit of the present disclosure should not be limited to the described embodiments, and not only the claims described later, but also all modifications equivalent or equivalent to these claims belong to the scope of the present disclosure.

1. A close-contact type nerve stimulator, comprising:
a fixed part disposed across front and rear of an ear;
a body part rotatably connected with respect to the fixed part;
an electrode part installed on the body part and configured to contact auricle or external auditory meatus; and
a control part configured to adjust an angle of rotation of the body part with respect to the fixed part,
wherein the electrode part comes into close contact with a surface of the auricle or the external auditory meatus by a rotation of the body part so as to stimulate vagus nerve distributed in the auricle or the external auditory meatus.

2. The close-contact type nerve stimulator of claim 1, wherein the control part comprises:
a first adjustment member provided as a shaft that hinge couples the fixed part and the body part;
a second adjustment member disposed on either one of the fixed part or the body part, and configured to slide toward a remaining one; and
a third adjustment member disposed on a remaining one of the fixed part or the body part, and famed to come into contact with the second adjustment member,
wherein the third adjustment member rotates, by a sliding of the second adjustment member, one of the fixed part and the body part relative to the remaining one with the first adjustment member as a center.

3. The close-contact type nerve stimulator of claim 2, wherein the third adjustment member comprises:
an internal space in which a portion of the second adjustment member is accommodated; and
an inclined surface to which a portion of the second adjustment member contacts,
wherein the portion of the second adjustment member is slidable along the inclined surface, and the inclined surface is rotated around the first adjustment member.

4. The close-contact type nerve stimulator of claim 3, wherein when the second adjustment member moves from one of the fixed part and the body part toward the remaining one, an angle between the fixed part and the body part decreases, whereas when the second adjustment member moves from the remaining one of the fixed part and the body part toward the one, the angle between the fixed part and the body part increases.

5. The close-contact type nerve stimulator of claim 1, wherein the body part is capable of elastically restoring to an original position thereof.

6. The close-contact type nerve stimulator of claim 1, wherein the electrode part comprises a first electrode in contact with the external auditory meatus,
wherein the first electrode is flexibly deformed according to a size or shape of the external auditory meatus so as to be in close contact with the surface of the external auditory meatus.

7. The close-contact type nerve stimulator of claim 6, wherein the first electrode comprises:
a contact member in contact with the external auditory meatus; and
deformable members configured to form spaces at regular intervals outside the contact member,
wherein the deformable members are provided to be stretchable when the contact member contacts the external auditory meatus by the rotation of the body part, and
wherein the contact member is flexibly deformed according to the shape of the external auditory meatus by an expansion and contraction of the deformable members, and comes into close contact with the surface of the external auditory meatus.

8. The close-contact type nerve stimulator of claim 6, wherein the electrode part further comprises a second electrode installed at a position on the body part contactable with tragus.

9. The close-contact type nerve stimulator of claim 6, wherein the electrode part further comprises a third electrode installed at a position on the body part contactable with cavum concha.

10. The close-contact type nerve stimulator of claim 6, wherein the electrode part further comprises a fourth electrode installed at a position on the body part contactable with cymba concha.

11. A close-contact type nerve electrode, comprising:
a body part located in auricle;
a first electrode installed at a position on the body part contactable with external auditory meatus;
a second electrode installed at a position on the body part contactable with tragus;
a third electrode installed at a position on the body part contactable with cavum concha; and
a fourth electrode installed at a position on the body part contactable with cymba concha,
wherein at least one of the first electrode, the second electrode, the third electrode, or the fourth electrode is flexibly deformed according to a size or shape of the auricle or the external auditory meatus, and comes into close contact with a surface of the auricle or the external auditory meatus to effectively stimulate vagus nerve.

12. The close-contact type nerve electrode of claim 11, wherein the at least one of the first electrode, the second electrode, the third electrode, or the fourth electrode comprises:
a contact member in contact with the auricle or the external auditory meatus; and
deformable members configured to form spaces at regular intervals outside the contact member,
wherein the deformable members are provided to be stretchable when the contact member contacts the auricle or the external auditory meatus by a rotation of the body part, and
wherein the contact member is flexibly defamed according to the shape of the auricle or the external auditory meatus by an expansion and contraction of the deformable members, and comes into close contact with the surface of the auricle or the external auditory meatus.
